(19) United States
(12) Patent Application Publication
(10) Pub. No.: US 2014/0103133 A1
(43) Pub. Date: Apr. 17, 2014

(54) Web-Material Dispenser With Air Freshener

(71) Applicants: Todd Muderlak, Whitefish Bay, WI (US); Deborah Ponath, Waukesha, WI (US)

(72) Inventors: Todd Muderlak, Whitefish Bay, WI (US); Deborah Ponath, Waukesha, WI (US)

(21) Appl. No.: 14/054,326

(22) Filed: Oct. 15, 2013

Related U.S. Application Data
(60) Provisional application No. 61/713,845, filed on Oct. 15, 2012.

(51) Int. Cl. A47K 10/34; A61L 9/12

(52) U.S. Cl. CPC A47K 10/34; A61L 9/12
USPC 239/6; 242/598.1

(57) ABSTRACT

A dispenser for dispensing web material from a roll contained within the dispenser includes an air freshener for providing a pleasing fragrance to the area surrounding the dispenser. The dispenser may include a housing to which the air freshener may be attached. The housing may include a pivoting cover, and the air freshener may be coupled to the pivoting cover to selectively cover one of the rolls of web material housed within the dispenser. The air freshener may alternatively be supported within a mandrel configured to support one of the rolls of web material within the dispenser. A fan may be provided and configured to push the fragrance into the surrounding environment.

### CROSS REFERENCE TO RELATED APPLICATIONS

The present application claims priority to U.S. Provisional Patent Application Ser. No. 61/713,845 filed on Oct. 15, 2012 and entitled Web-Material Dispenser with Air Freshener, the entirety of which is hereby incorporated by explicit reference thereto.

### BACKGROUND OF THE INVENTION

This invention relates generally to dispensers for dispensing sheet material. More particularly, this invention relates to a roll dispenser for dispensing sheet material and having an air freshening device incorporated in the dispenser

Dispensers for rolls of flexible sheet material, such as paper toweling and toilet paper, have been employed for a great many years. Dispensers for web material typically include a housing defining a cavity in which the rolls of web material are rotatably secured. The housing defines an opening through which the user of the dispenser may access the web material for dispensing thereof. The housing typically rotatable supports one or more mandrels about which the rolls of web material are rotatably secured for advancement thereof.

It is generally known to provide a restroom or other area in which the dispenser is deployed with a fragrance to provide the surrounding area with a pleasant scent. Some dispensers have employed housings or mandrels that include an air freshening device or element to thereby provide a pleasing fragrance to the surrounding area. However, known dispensers suffer from a number of disadvantages. For instance, in known dispensers having an air freshener element, it is typically difficult to access the air freshener element for replacement thereof. Some known dispensers are configured so that user-directed interaction with the dispenser is required to activate the emission of the fragrance, which results in excess fragrance emission or in too little fragrance being emitted to effectively provide a desirable scent to the surrounding area.

Accordingly, a need exists for a web material dispenser including an air freshening device that is relatively simple, requires little maintenance, and effectively delivers a consistent amount of fragrance to the surrounding area.

### SUMMARY OF THE INVENTION

The present invention provides a dispenser for dispensing web material from roll contained within the dispenser. The dispenser includes an air freshener for providing a pleasing fragrance to the area surrounding the dispenser. The air freshener may be secured to a portion of the housing of the dispenser. In one embodiment, the air freshener is secured to a pivoting cover that is configured to selectively cover one of the rolls of web material. In another embodiment, the air freshener is secured within a mandrel supporting one of the rolls of web material. In yet another embodiment of the invention a fan is incorporated to “push” the fragrance into the ambient air.

Various other features, objects and advantages of the invention will be made apparent from the following description taken together with the drawings.

### DESCRIPTION OF THE DRAWINGS

FIG. 1 is an isometric view of a web material dispenser according to one construction of the present invention;

FIG. 2 is an isometric view of the web material dispenser with a front portion of the web material dispenser removed;

FIG. 3 is a front elevation view of the web material dispenser of FIG. 2;

FIG. 4 is an end elevation view of the web material dispenser of FIG. 2;

FIG. 5 is a bottom plan view of the web material dispenser of FIG. 2;

FIG. 6 is an isometric view of an air freshener holder according to the invention;

FIG. 7 is an isometric view of a web material dispenser according to another construction of the invention;

FIG. 8 is a front elevation view of the web material dispenser of FIG. 7 with a front portion of a housing removed;

FIG. 9 is an isometric view of the web material dispenser as shown in FIG. 8 with one of the rolls of web material removed;

FIG. 10 is an end elevational cross-section view through the web material dispenser of the present construction;

FIG. 11 is an isometric view of another construction of the web material dispenser of the present invention;

FIG. 12 is a rear elevation view of the dispenser of FIG. 11;

FIG. 13 is an end elevation view of the dispenser of FIG. 11;

FIG. 14 is a partially exploded isometric view of the dispenser of FIG. 11;

FIG. 15 is an isometric view of yet another construction of the web material dispenser of the present invention;

FIG. 16 is a rear elevation view of the dispenser of FIG. 15

FIG. 17 is an end elevation view of the dispenser of FIG. 15; and

FIG. 18 is a partially exploded isometric view of the dispenser of FIG. 15.

### DETAILED DESCRIPTION OF THE PREFERRED EMBODIMENTS

As will be described more fully herein, the present invention is directed to a roll dispenser, such as a toilet paper dispenser, having an air freshener coupled to or otherwise arranged with the roll dispenser.

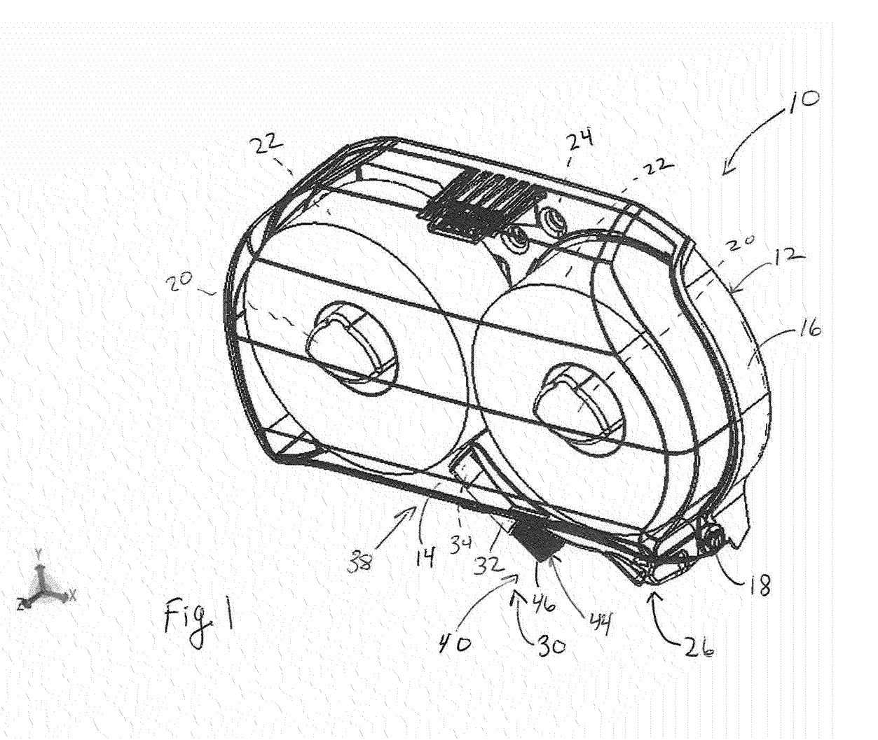

Referring now to the Drawings, and initially to FIG. 1, a roll dispenser 10 includes a housing 12 having a front portion 14 and a rear portion 16 coupled to one another to define a cavity in which a pair of rolls of web material are rotatably mounted as will be described herein. The front portion 14 may be transparent so that a user may determine whether the dispenser 10 needs refilling. The rear portion 16 may be generally opaque or may be transparent like the front portion 14. The housing 12 defines an opening at a lower end thereof through which the user may grasp the roll of web material for dispensing thereof. The front portion 14 may be configured to be removably coupled to the rear portion 16 so the user may selectively access the cavity for changing the roll of web material as desired. The front portion 14 may be releasably coupled to the rear portion 16 by a pair of longitudinally spaced fasteners 18 configured to be received through aligned holes (not shown) in the respective front and rear portions 14 and 16.

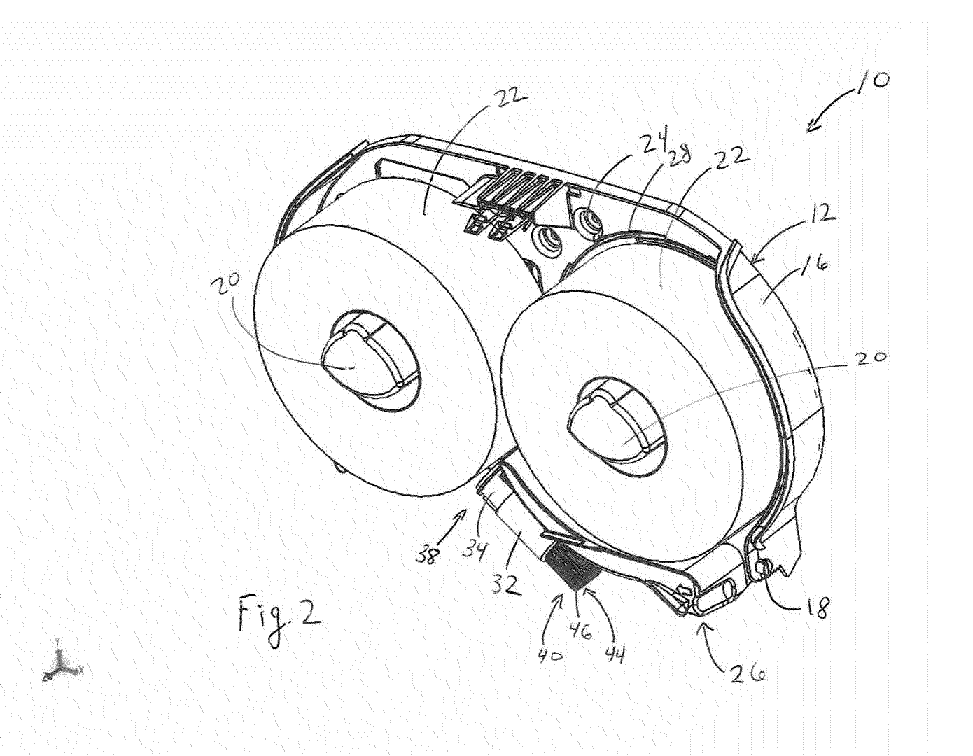

With additional reference now to FIG. 2, the dispenser 10 is shown with front portion 14 removed. The dispenser 10 includes a pair of mandrels 20 rotatably coupled to the rear portion 16. Each of the mandrels 20 is configured to support a roll 22 of web material such as, e.g., toilet paper, paper towel, etc. The mandrels 20 may have a generally tri-lobular shape as illustrated or may take on any other shape capable of securely rotatably supporting the rolls 22. The rear portion 16 may include one or more apertures 24 configured to receive a fastener to couple the rear portion 16 and thereby the dispenser 10 to a wall or other surface as desired. The dispenser 10 may additionally include a pivoting door 26 configured to cover one of the rolls 22 when the other of the rolls 22 is available for dispensing. When the other of the rolls 22 is exhausted, the door 26 may then be slid out of the way of the roll 22 for dispensing thereof. In particular, the pivoting door 26 may be slidably disposed within a track 28 formed over a portion of the rear portion 16 so the door 26 may be slidably moved along the track 28 from a deployed position in which the door 26 covers the roll 22 and a second position in which it is moved out of the way of the roll 22 for dispensing of the web material. In one embodiment, the track 28 has a generally arcuate shape so that the door 26 is moved to a position disposed between the two mandrels 20 so as to not interfere with the dispensing of the rolls of web material as is generally understood.

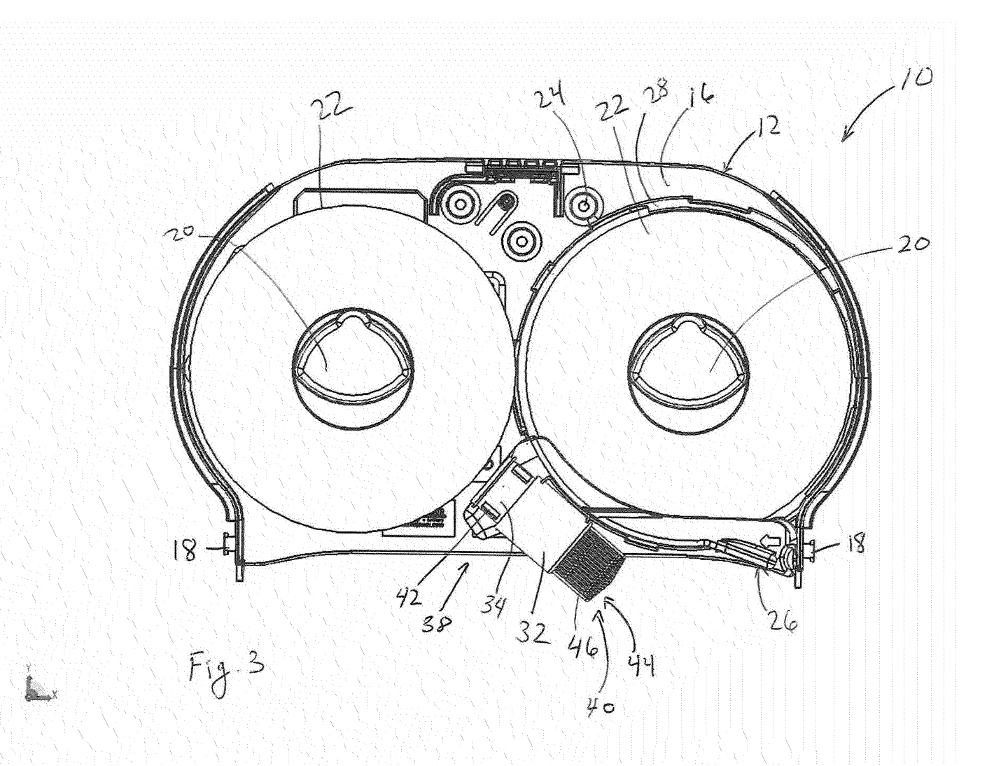

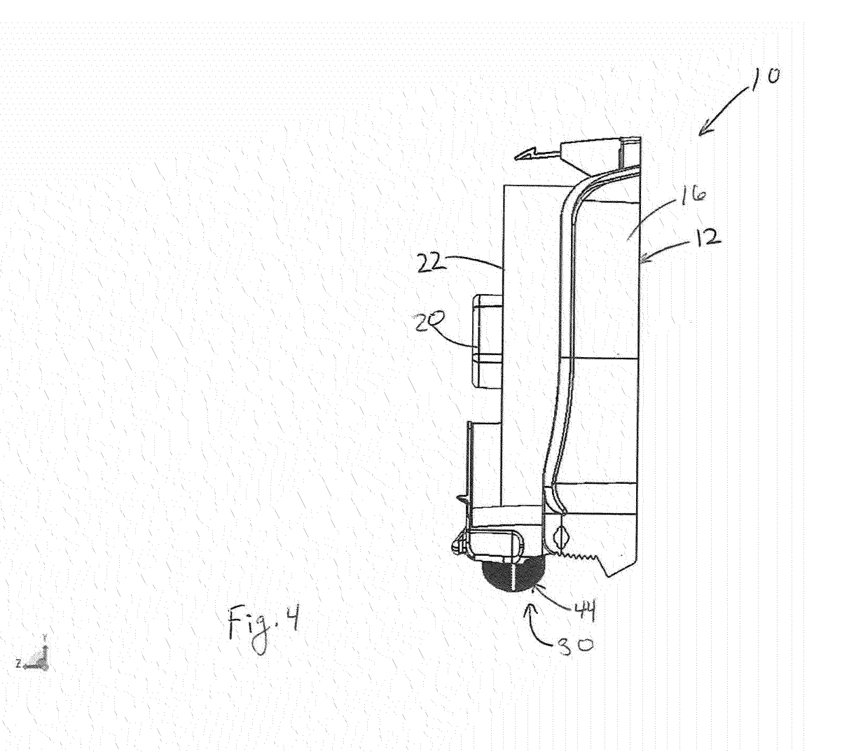

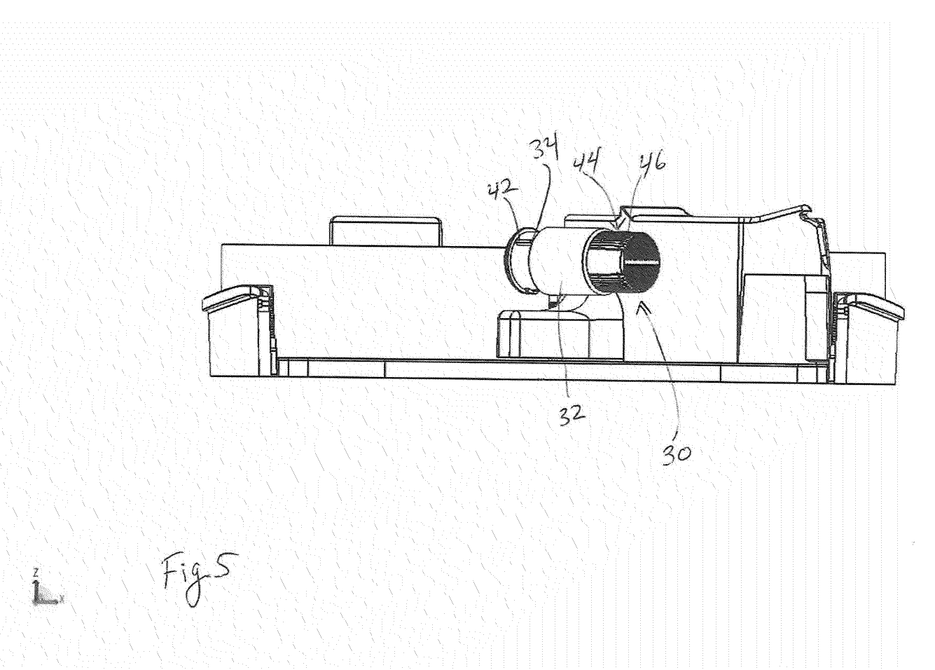

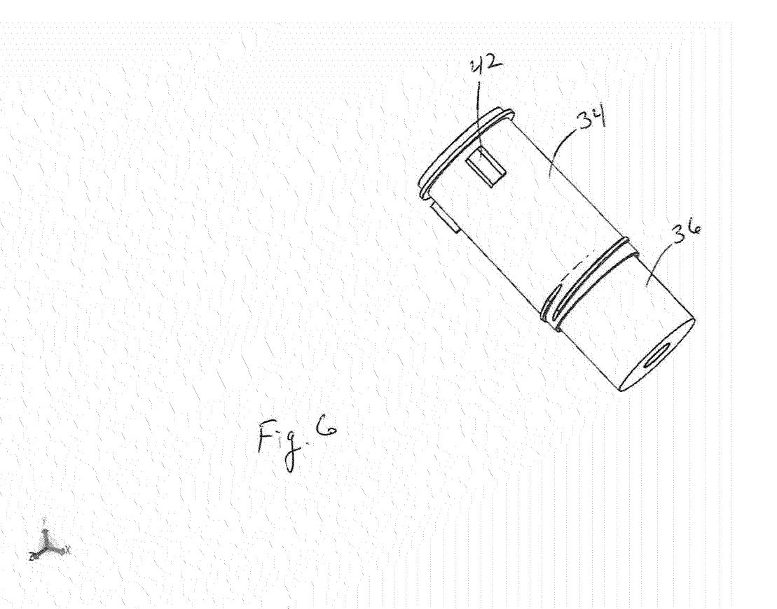

With additional reference to FIGS. 3-6, the dispenser 10 includes an air freshener assembly 30. Air freshener assembly 30 includes an air freshener housing 32, an air freshener holder 34, and a deodorant element 36. The air freshener housing 32 may be cylindrically shaped or have any other suitable shape and is coupled to the housing 12 of the dispenser 10. The air freshener housing 32 defines a cavity configured to removably receive the air freshener holder 34. The air freshener holder 34 may be correspondingly shaped to be received within the cavity and include means for securing the air freshener holder 34 within the cavity of the air freshener housing 32. The air freshener holder 34 includes a first end 38 opposite a second end 40. The first end 38 may include a number of tabs, clips or other fastener 42 adjacent the first end 38 configured for securing the air freshener holder 34 to the air freshener housing 32.

The second end 40 includes an opening (not shown) configured to selectively receive the deodorant element 36. The deodorant element 36 may be rotatably coupled to the air freshener holder 34 so that as desired, the deodorant element 36 may be rotatably removed from the air freshener holder 34 for discarding and replacement as may be desired. In this manner, when the deodorant element 36 loses its fragrance over time, it maybe replaced by a refill as desired by the user. The deodorant element 36 is secured within an end 44 of the air freshener housing 32. The end 44 may include a number of slits or apertures 46 to enable the scent of the deodorant element 36 to permeate from the air freshener assembly 30 to make the surrounding area fragrant. The end 44 of the air freshener housing may be selectively removable as desired for obtaining access to the deodorant element 36 for replacement thereof.

The air freshener assembly 30 may be positioned at an oblique angle relative to the dispenser 10. More particularly, the air freshener assembly 30 may be at least partially disposed between the rolls 22 so as to not interfere with the rotation of the rolls 22 and the dispensing of the web material. In one embodiment, the air freshener assembly 30 may be coupled to the pivoting door 26. In this manner, when the pivoting door 26 is in the deployed position, at least a portion of the air freshener assembly 30 may extend below the dispenser 10. When the pivoting door 26 is moved to its second position, the air freshener assembly 30 is carried along with the pivoting door 26 and moved upward within the cavity defined by the housing 12. Understandably, the air freshener assembly 30 may be disposed at any number of alternative locations within the dispenser as long as the position does not interfere with the dispensing of the web material.

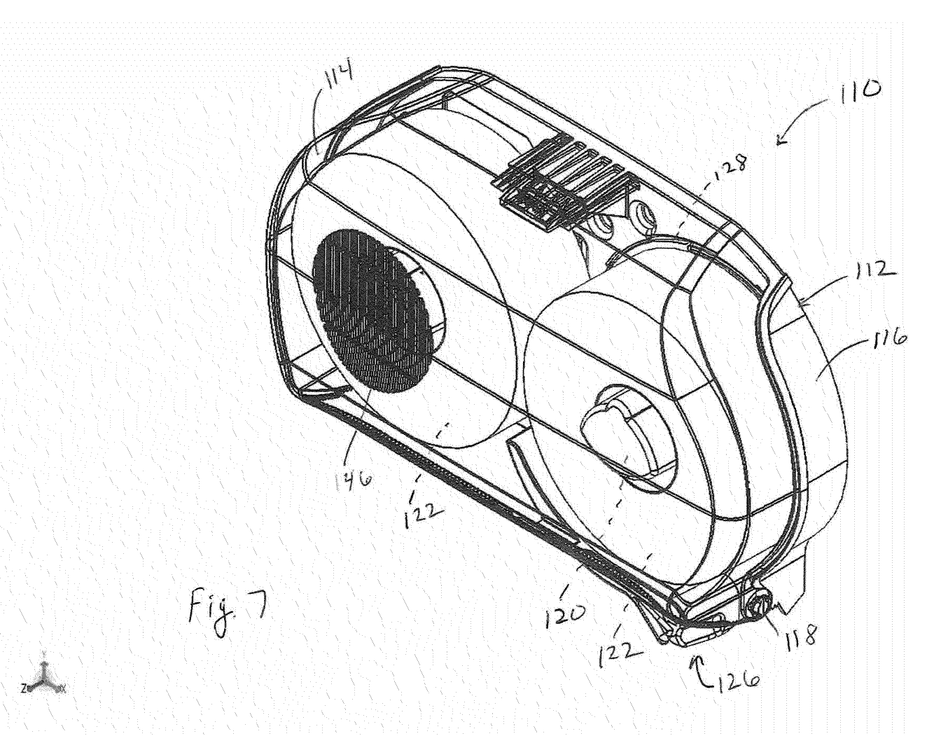

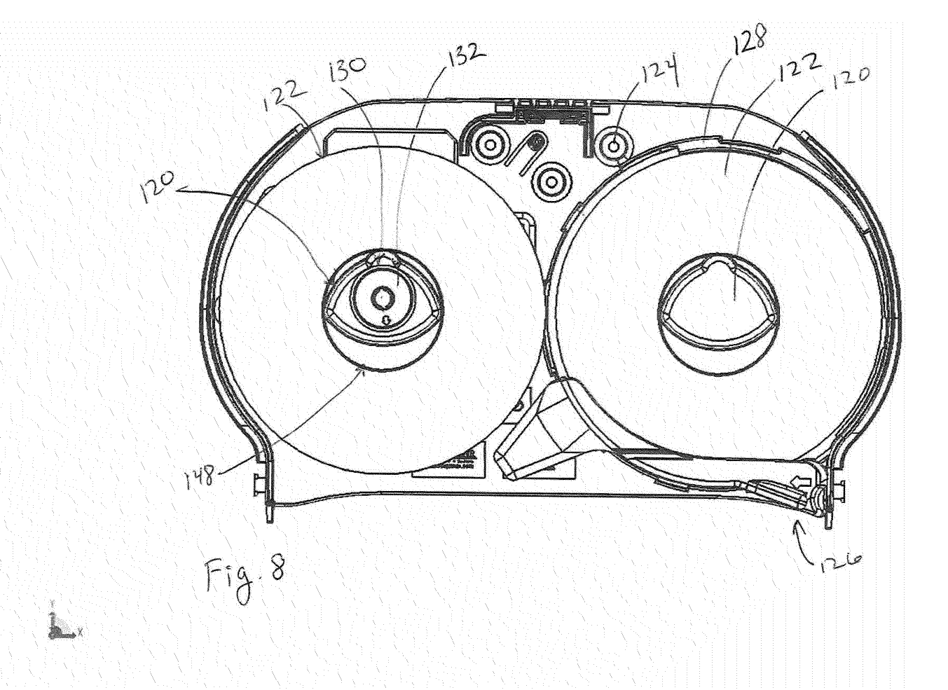

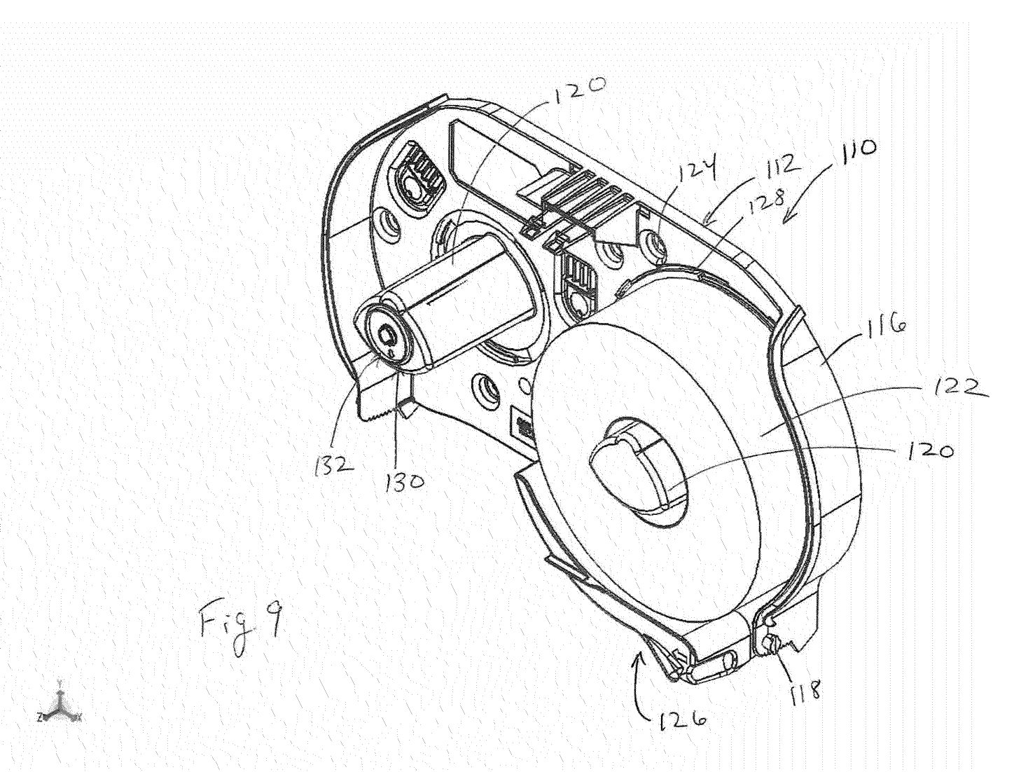

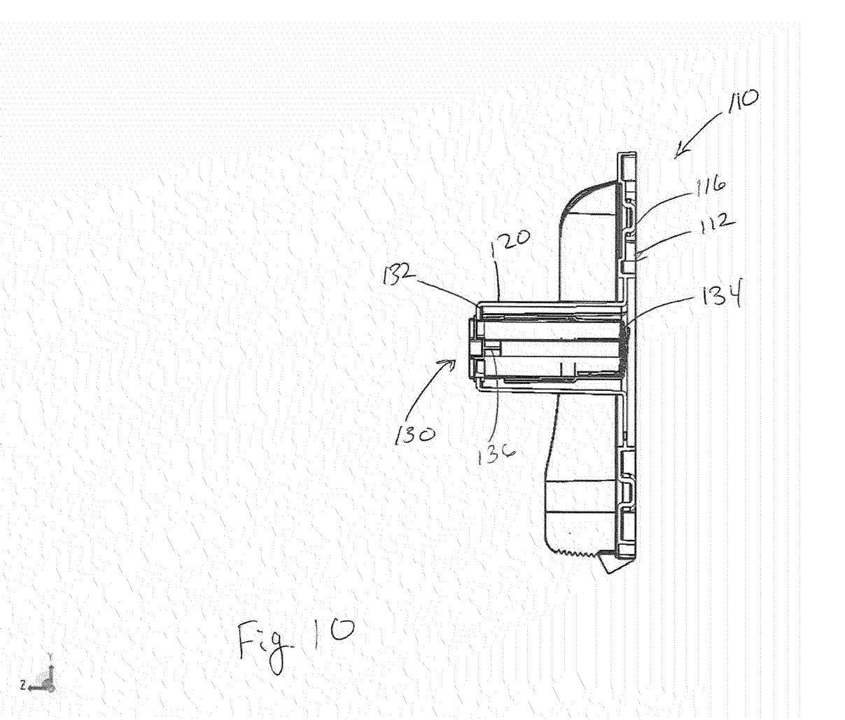

Referring now to FIGS. 7-10, an alternative construction of the dispenser 110 is illustrated. The dispenser 110 of the present construction may operate in a substantially similar manner to the dispenser 10 previously described. The dispsner 110 has a housing 112 including a front portion 114 and rear portion 116 connected to one another to define a cavity for rotatably receiving rolls 22 of web material such as toilet paper or paper towel. The front and rear portions 114 and 116 may be secured to one another by a pair of opposing fasteners 118 received through aligned apertures (not shown) in each of the front and rear portions 114 and 116. The front and rear portions 114 and 116 define an opening at a lower end thereof to define a dispensing area whereby the user of the dispenser 10 may access the web material to be dispensed. The rear portion 116 may include one or more apertures 124 fir receiving fasteners to couple the dispenser 10 to a wall or other structure.

The dispenser 110 includes a pair of spaced mandrels 120 for rotatably supporting corresponding rolls 122 of web material. The mandrels 120 may be constructed in a manner substantially similar to mandrels 20. The rolls 122 are rotatable about the mandrels 120 for advancing of the web material for dispensing as previously described with respect to mandrels 20. As in dispenser 10, the dispenser 110 includes a pivoting door 126 that is movable along a track 128 to selectively expose one of the rolls 122 for dispensing thereof when the other of the rolls 122 has been exhausted. The pivoting door 126 operates in the same manner described with respect to dispenser 10.

At least one of the mandrels 120 may be configured to receive an air freshener assembly 130. In particular, the mandrel 120 may include an open end 148 in which the air freshener assembly 130 may be received. The mandrel 120 may be sized and shaped for securely receiving the air freshener assembly 130 therein. In one construction, the mandrel 120 may include means to enable securing the air freshener assembly 130 therein. In the alternative, the air freshener assembly 130 may be configured to be friction fit within the interior of the mandrel 120 to secure the air freshener assembly 130 therein. In one construction, both mandrels 120 may be configured to receive an air freshener assembly 130.

The air freshener assembly 130 may be similarly constructed to the air freshener assembly 30 and include an air freshener housing 132 that receives an air freshener 134, which in turn receives a selectively replaceable deodorant element 136. The deodorant clement 136 may be positioned to be near the outer edge of the mandrel 120 for emitting the fragrance to the surrounding area.

The front portion 114 of the present construction may include a number of apertures, slits, or similar openings 146. The openings 146 are configured to enable the fragrance from the deodorant element 136 to be emitted to the surrounding area. The openings 146 may be disposed on the face of the front portion 114 but may also be disposed elsewhere as desired.

A fan assembly, not shown, may be included with the air freshener assembly 130 or mounted within the rear portion of the mandrel 120. As the fan of the fan assembly spins, it passes air over the air freshener 134 to push scent into the ambient air through openings 146.

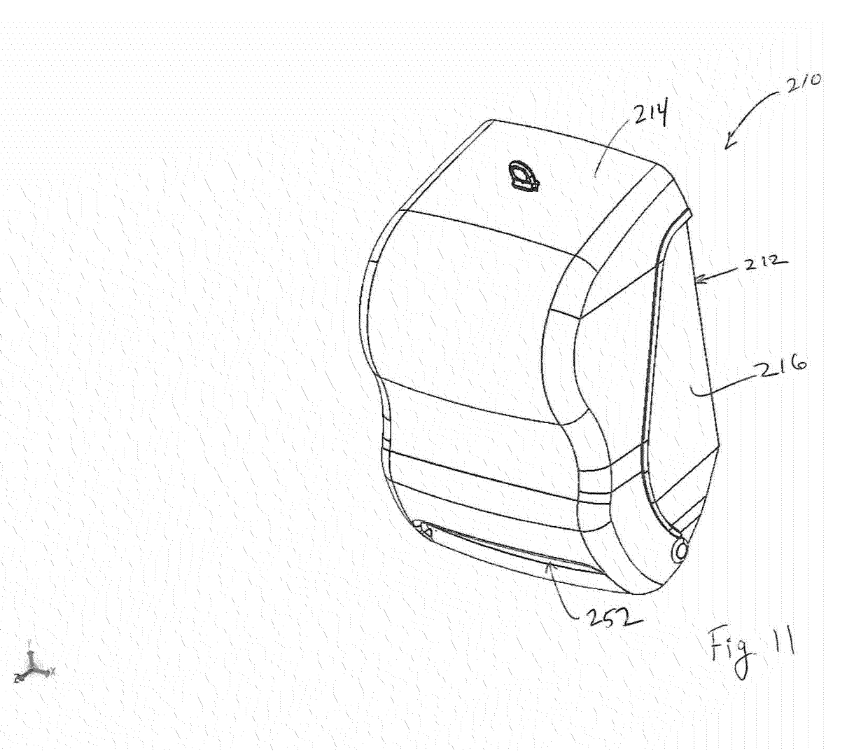

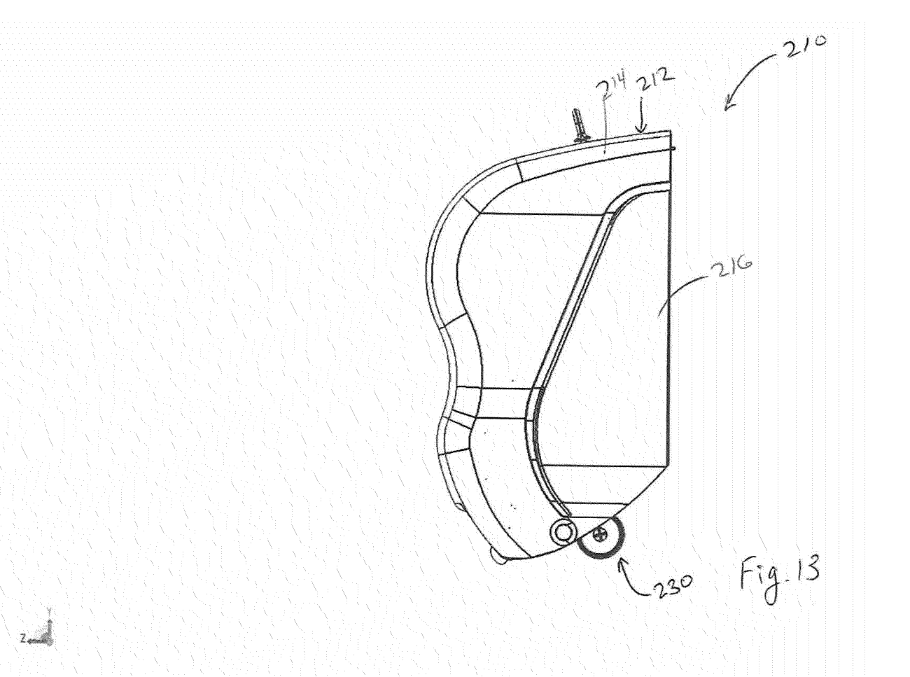

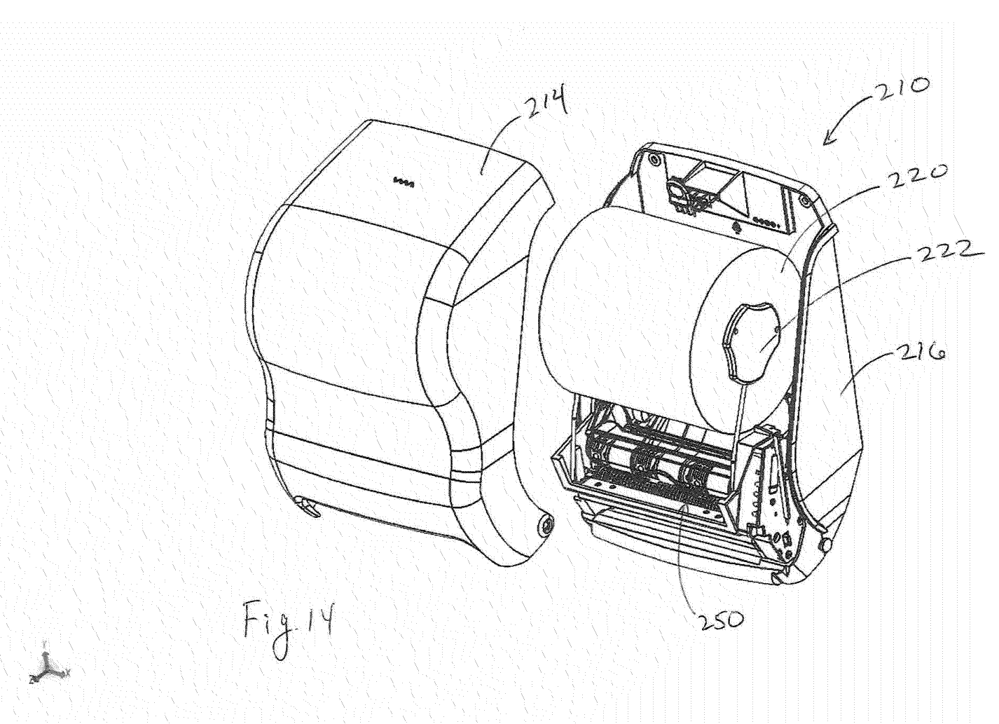

Turning now to FIGS. 11-14, another embodiment of the web material, dispenser 210 is illustrated in which the dispenser 210 is a paper towel dispenser or the like. The dispenser 210 may be any type of paper towel dispenser such as an automatic dispenser in which the paper towel or other web material is dispensed upon activation of a sensor such as a proximity sensor of the kind known in the art. In the alternative, the dispenser 210 may include a lever or similar element configured to be engaged by the user for advancing the roll of web material for dispensing thereof.

With continuing reference to FIGS. 11-14, the dispenser 210 includes a housing 212 including a front portion 214 and a rear portion 216 defining a cavity for receiving a roll 220 of web material that is rotatably supported by a pair of aims 222 on opposing lateral sides of the dispenser 210 and supported by a chassis 250. The roll 220 of web material is selectively advanceable to be dispensed through an opening 252 in the front portion.

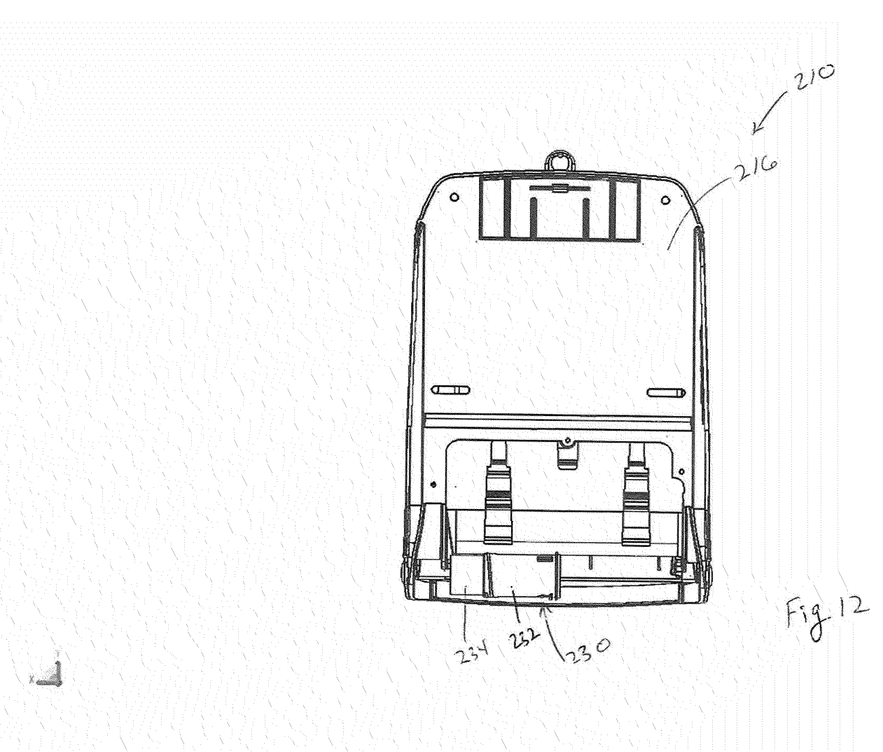

With particular reference now to FIG. 12, an air freshener assembly 230 is mounted to the rear portion 216 of the dispenser 210. In this manner, the air freshener assembly 230 may be configured to deliver a pleasing fragrance to the area surrounding the dispenser 210. The air freshener assembly 230 may be configured substantially similar to air freshener assemblies 30 and 130 and include an air freshener housing 232 and air freshener holder 234 configured to selectively hold an air freshener element (not shown) like that previously described. The air freshener assembly 230 may be accessed through the chassis 250 after removal of the roll 220 of web material to selectively change the air freshener element as may be desired.

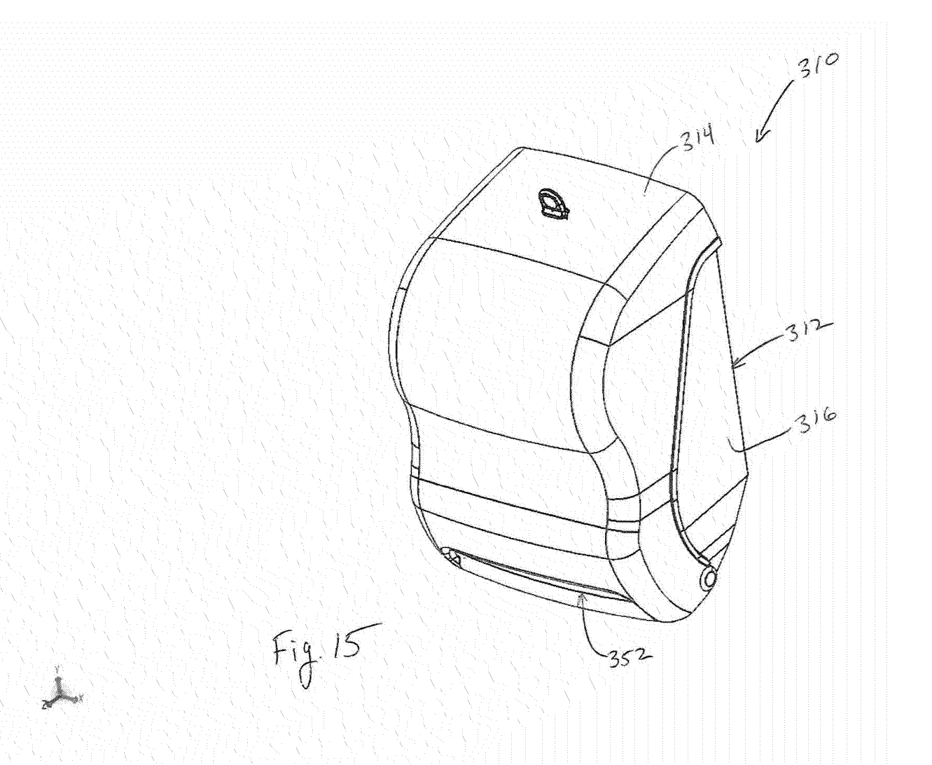

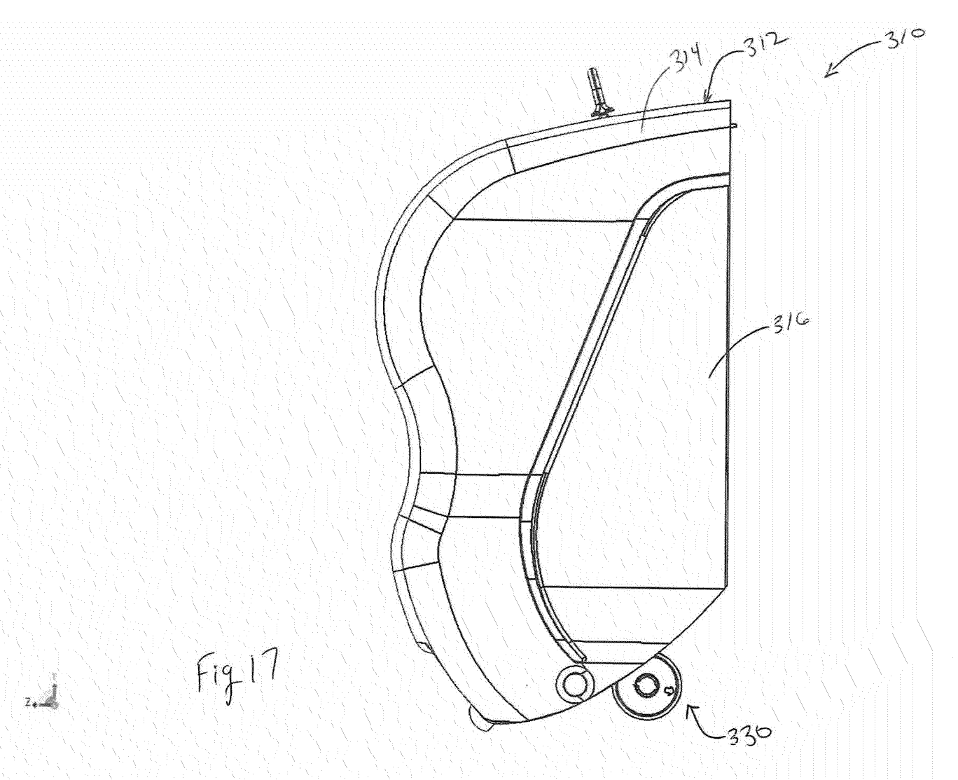

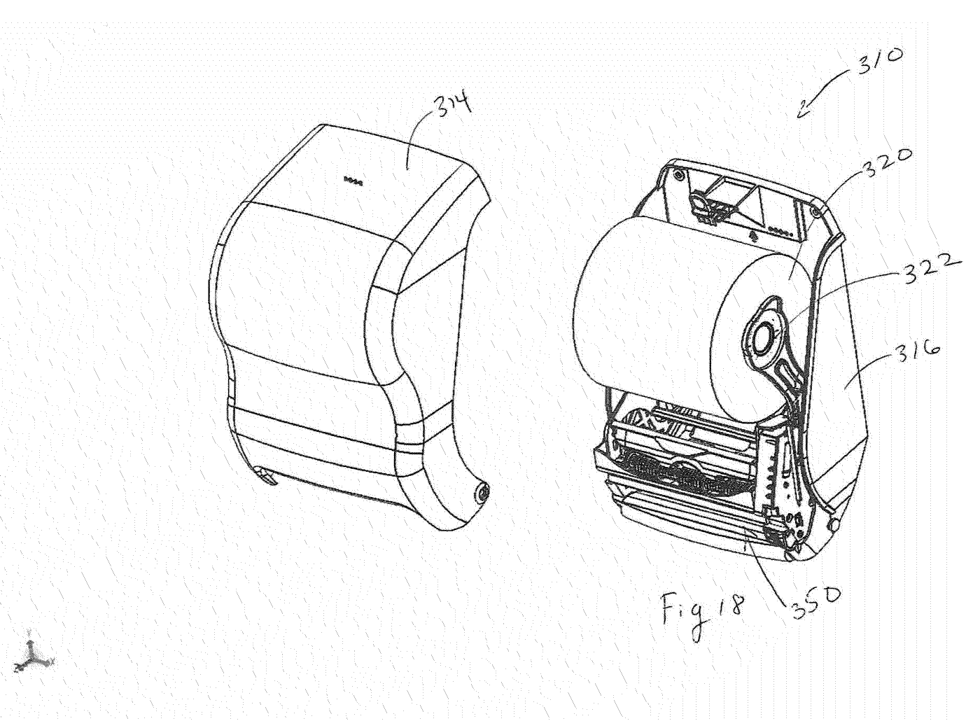

Now referring to FIGS. 15-18, another embodiment of a web material dispenser 310 is illustrated. Like the embodiment of FIGS. 11-14, the dispenser 310 may be a paper towel dispenser in the form of an automatic or manually activated dispenser. The dispenser 310 includes a housing 312 having a front portion 314 and a rear portion 316 defining a cavity for rotatably receiving a roll 320 of web material such as e.g., paper towel. The roll 320 is rotatably supported by a pair of laterally opposing arms 322 supported by a chassis 350.

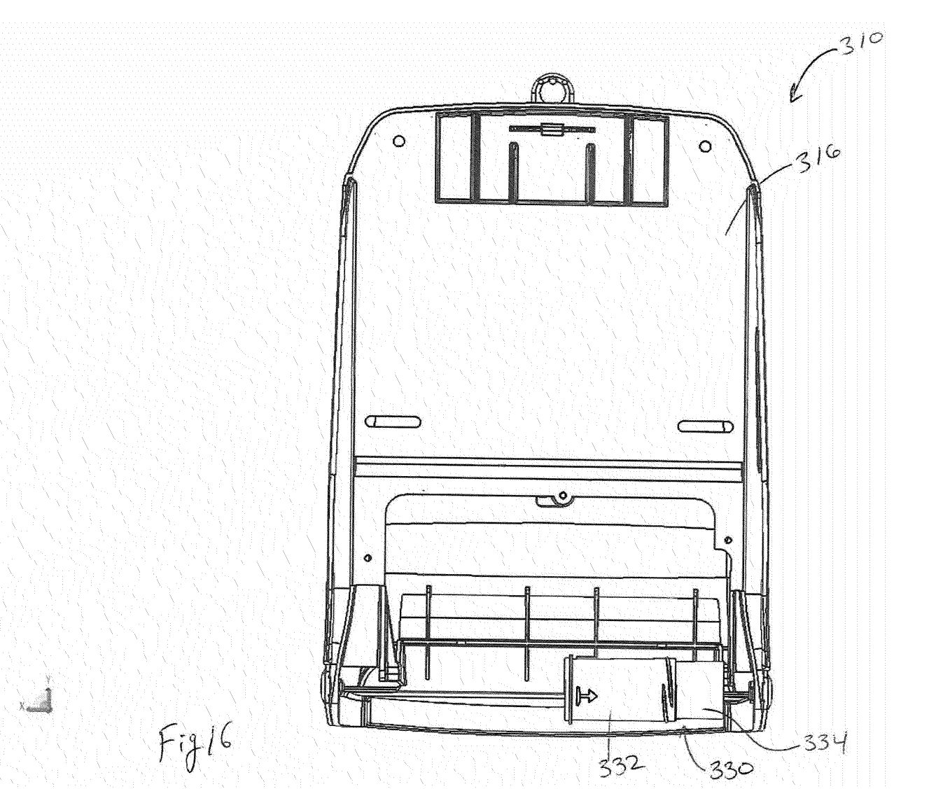

With particular reference now to FIG. 16, an air freshener assembly 330 is mounted to the rear portion 316 of the housing 312. The an freshener assembly 330 may include an air freshener housing 332 and an freshener holder 334 for removably holding an air freshener element similar to an freshener assemblies 30, 130, and 230. As in the previous embodiment, the air freshener assembly 330 may be accessed through the chassis 350 when the roll 320 of web material is removed.

The dispenser of the present invention may have other applications aside from use in paper towel rolls and paper toweling. Although the invention has been herein shown and described in what is perceived to be the most practical and preferred embodiments, it is to be understood that the invention is not intended to be limited to the specific embodiments set forth above. Rather, it is recognized that modifications may be made by one of skill in the art of the invention without departing from the spirit or intent of the invention and, therefore, the invention is to be taken as including all reasonable equivalents to the subject matter of the appended claims.

We claim:

1. A web material dispenser comprising:
at least one mandrel coupled to the housing configured to rotatably support a roll of web material; and
an air freshener assembly coupled to the dispenser and configured to emit a fragrance to an area surrounding the dispenser.

2. The web material dispenser of claim 1, further comprising a housing defining a cavity in which the roll of web material is rotatably supported and wherein the at least one mandrel is coupled to the housing.

3. The web material dispenser of claim 1, wherein the at least on mandrel comprises a pair of spaced mandrels each configured to rotatable support a roll of web material.

4. The web material dispenser of claim 1, further comprising a pivoting cover configured to be pivotally moved between a deployed position in which the web material cannot be dispensed and a second position in which the web material can be dispensed.

5. The web material of claim 4, wherein the air freshener assembly is coupled to the pivoting cover.

6. The web material dispenser of claim 1, wherein the air freshener assembly comprises,
an air freshener housing defining a cavity;
an air freshener holder receivable within the cavity; and
a deodorant element removably coupled to the air freshener holder.

7. The web material dispenser of claim 1, wherein the air freshener is disposed at an oblique angle relative to the dispenser.

8. The web material dispenser of claim 1, wherein the at least one mandrel defines a cavity therein and the an freshener assembly is removably received within the cavity.

9. The web material dispenser of claim 8, further comprising a housing having a plurality of apertures over a portion thereof and aligned with the mandrel for emitting a fragrance of the air freshener.

10. A dispenser for a web material, the dispenser comprising:
a housing defining a dispensing area fir the dispensing of the web material;
at least one mandrel rotatably coupled to the housing and configured to support a roll of the web material;
an air freshener assembly coupled to the dispenser and positioned adjacent the roll of web material.

11. The dispenser of claim 10, wherein the at least one mandrel comprises a first and second mandrel longitudinally spaced from one another, each of the first and second mandrels configured to support a corresponding first and second roll of web material, and wherein the air freshener is disposed between the first and second roll of web material.

12. The dispenser of claim 10, wherein the at least one mandrel defines a cavity in which the air freshener assembly is received.

13. The dispenser of claim 12, wherein the housing includes a plurality of apertures aligned with the at least one mandrel and configured to enable emission of a fragrance from the air freshener assembly.

14. The dispenser of claim 10, wherein the air freshener assembly comprises,
an air freshener housing defining a cavity;
an air freshener holder secured within the cavity of the air freshener housing; and
a deodorant element removably coupled to the air freshener holder.

15. The dispenser of claim 10, further comprising a pivoting cover coupled to the housing and configured to be moved between a deployed position and a second position and wherein the air freshener assembly is coupled to the pivoting cover.

16. A method of providing a fragrance to an area surrounding a web material dispenser, the method comprising the steps of:
coupling an air freshener assembly to the web material dispenser, wherein the dispenser includes first and second mandrels longitudinally spaced from one another and configured to rotatably support a roll of web material.

17. The method of claim 16, further comprising the step of positioning the air freshener between the first and second mandrels at an oblique angle relative to the dispenser.

18. The method of claim 17, further comprising the step of positioning the air freshener within a cavity defined by one of the first and second mandrels.
